(19) United States
(12) Patent Application Publication
(10) Pub. No.: US 2019/0192064 A1
(43) Pub. Date: Jun. 27, 2019

(54) MICRO-DEVICE AND SYSTEM FOR DETERMINING PHYSIOLOGICAL CONDITION OF CERVICAL TISSUE

(71) Applicant: MB Device LLC, Louisville, KY (US)

(72) Inventors: Divya Cantor, Louisville, KY (US); Barry Simon, Lexington, KY (US); David Phelps, Louisville, KY (US); Vasiliy Abramov, Louisville, KY (US); John Vanderburgh, lLouisville, KY (US)

(73) Assignee: MB Device LLC, Louisville, KY (US)

(21) Appl. No.: 16/292,579

(22) Filed: Mar. 5, 2019

Related U.S. Application Data
(63) Continuation-in-part of application No. 15/333,248, filed on Oct. 25, 2016, now abandoned.
Application No. 15/333,248 is a continuation-in-part of application No. 13/611,265, filed on Sep. 12, 2012, now abandoned.

(51) Int. Cl. A61B 5/00; A61B 5/053; H01Q 7/00; A61B 5/1473

(52) U.S. Cl. CPC A61B 5/435; A61B 5/0538; A61B 5/0002; A61B 2562/16; A61B 5/6847; A61B 5/1473; A61B 2560/0214; H01Q 7/00

(57) ABSTRACT

A micro-device and system for monitoring cervical tissue includes micro-circuitry for simulating electrochemical impedance spectroscopy and a system for monitoring the physiological condition of the cervical tissue.

### FIELD OF THE INVENTION

This invention relates to the treatment of pregnant mammals and more particularly to a device and system useful for determining the physiological condition of cervical tissue.

### BACKGROUND OF THE INVENTION

The birthing process in mammals has a normal or average duration, for example human gestation is normally 266 days while for horses it normally ranges between 335 and 345 days. For cattle gestation averages 283 days while for swine about 113 to 116 days and for sheep 144 to 151 days. The actual period for human gestation is variable but is considered full-term to conclude between 38-42 weeks post-conception. In humans, for example, labor and birth that commences earlier than 36 weeks as a result of a high-risk pregnancy or other complications presents serious if not fatal problems for the newborn infant that can well continue on to adulthood. Delivery at 30-32 weeks normally necessitates considerable time in an NICU and delivery very early at around 23 weeks is considered almost uniformly fatal

For most domestic animals entering into labor and the birthing process occurs with little assistance. However, for humans in particular and certain valuable domestic animals, such as thoroughbred horses, the gestation period in the final trimester is normally closely monitored for the beginning of labor and for complications such as preterm labor resulting in preterm birth.

For domestic animals a preterm birth will usually produce a still born or a weak and sickly animal requiring expensive attention. Especially in the case of thoroughbred horses where stud fees may run up to six figures a premature foal may not survive or perform to the expectations of the breeder resulting in a substantial financial loss.

For humans the societal burden of preterm births in the United States was estimated to be $26 billion in 2005 by the Committee on Understanding Premature Birth and assuring Healthy Outcomes under the auspices of the US Institute of Medicine. These costs include medical care services at $16.9 billion; maternal delivery costs $1.9 billion; early intervention services $611 million; special education services associated with a high incidence of disabling conditions associated with premature infants added $1.1 billion. Finally, the committee estimated that lost household and labor market productivity associated with those disabling conditions contributed $5.7 billion.

High-risk pregnancies are not always detected early nor easily determined in advance so that treatment can be provided to alleviate the harmful effects on the infant and mother of a preterm birth. Women pregnant for the first time or who may not have access to medical help can present a high-risk pregnancy without being aware of their condition. Early identification of high-risk pregnancies facilitates monitoring and prompt initiation of therapy. Early initiation of therapy has the potential to decrease the onset of labor, thus reducing the complications endured by the newborn as well as reducing the cost burden. However, monitoring of even normal pregnancies in the last trimester is also recommended if only to avoid unnecessary trips to the hospital because of false labor.

The human birthing process is divided into four stages. Stage 1 occurs from the onset of labor to full cervical dilation. Stage 2 is from full cervical dilation to delivery; stage 3 from delivery to expulsion of placenta and stage 4 from expulsion of the placenta to afterbirth recovery. Thus, monitoring the physiological parameters of the cervix in humans and mammals allows the clinician to follow the progress of a pregnancy during stage 1 of the process where complications, particularly preterm labor, can be recognized and possibly treated. In that connection some successful work has been done in this area but the methods employed require the presence of the clinician and usually employ invasive procedures.

It is recommended that monitoring of a pregnancy, especially in the last trimester of the pregnancy, be conducted frequently so that the onset of labor and any complications can be identified can be identified in the very early stages. In the case of domestic animals monitoring of the animal's pregnancy will normally require the on-site presence o f a veterinarian. For both animal and human patients this will be inconvenient.

In the case of human patients in the United States and other Western countries where medical services are more readily available, a pregnancy is normally monitored at the doctor's office as an outpatient or in the hospital. However, this requires a visit to the doctor's office or the hospital every few days in order to monitor the patient's progress. In developing countries, it may not be possible for a woman to see a doctor on a regular basis so that intervention to alleviate or treat a condition may be too late.

Various systems have been put forward to monitor the status of a pregnancy. For example, some systems employ small ultrasound reflectors signals transmitted by an ultrasound interface unit located outside of the body detect uterine contractions. For the measurement of uterine electrical activity, electro hysterography, has been proposed as an alternative approach for the detection of labor. For example, WO 94114373 (Garfield) records signals from embedded electrodes, WO 95/31932 and WO 96/39931 (Garfield) disclose a method which stores data and compares activity and U.S. Pat. No. 5,373,852 (Harrison) uses radio-telemetric transmission for sensing pressure, temperature and electrical activity. Rosenberg (WO 97/25922) discloses a further method for analysis of electromyographic data. These techniques relate exclusively to clinical settings, usually intrusive systems for data collection. U.S. Pat. No. 6,823,211 discloses a nonintrusive device including recording electrodes, a digital converter and a display unit that can be worn on the exterior of the abdomen or vaginal area of a body for the analysis of uterine electrical activity to monitor the progress of, and/or diagnose active labor and can be used to obtain an indication of uterine preparedness for labor in the initial phase of parturition before onset of active labor. Another system that relies on the measurement of uterine electrical activity is disclosed in U.S. Pat. No. 6,290,657 that, similar to the '211 patent, uses surface electrodes and a monitor to sense and analyze electrical signals that are transmitted by telephone to the physician's office.

In Patent Application Publication US 2009/0137925 the pregnancy is monitored by attaching a clip carrying a pair of tetra-pole electrodes to the patient's cervix. The electrodes that are electrically connected to a transducer cable that supplies the signal to an interface unit containing circuitry and a monitor. The transducer cable carries analog to digital conditioning hardware while the interface unit includes an algorithm for comparing typical impedance value with the impedance value of the cervix being measured. The resulting output can be a measure of time of labor vs. cervical stromal impedance (CSI) value or time in labor vs. cervical dilation and/or effacement for plotting a Friedman labor curve. The patient is immobilized during the procedure. US 20100/305406 disclose a gynecological device and mentions measuring impedance. However, this device is hard wired to a monitor or a transmitting device and is not designed for use while the patient is ambulatory.

A device suitable to be worn by the patient is shown in Patent Application Publication US2013/0053670. This device measures the potential developed by polarization and depolarization of vaginal and cervical tissue due to tissue contractions.

The prior art methods are inconvenient and are not suited for long term, continuous monitoring of the patient. In many cases monitoring involves the intervention of a clinician that requires the patient to visit the hospital or the clinician's office frequently to undergo the monitoring procedures. Placing sensors externally, as disclosed in several prior art publications, may introduce discrepancies in the data collected. For example, discrepancies due to variations in skin temperature and variations in contact pressure between the skin and the sensor. Other methods require that relatively large devices be placed in the patient that requires frequent removal to avoid odor and possible infection. Several methods require connection by wire to sensors at one end and to burdensome equipment at the output end that requires that the patient be non-ambulatory. In addition, many prior art systems are concerned only with the measurement of vaginal contractions which may be too late in the case of those at risk of premature birth.

Electrochemical Impedance Spectroscopy (EIS) is a useful and reliable method for determining the physiological condition of biological tissue. However, EIS employs alternating current to measure tissue potential which is then converted to impedance and plotted. The necessary circuitry is not readily miniaturized because it requires too may components and the power drain is too great for use in applications of the type to which the present invent applies.

### SUMMARY OF THE INVENTION

The present invention relates to a device using low power direct current to simulate alternating current in the manner of EIS. The device includes circuitry that is miniaturized for inclusion in devices that are adapted to be worn by the patient for extended periods of time while permitting the patient to be fully ambulatory during the monitoring procedure.

Accordingly, it is an object of the invention to monitor and record the physiological condition of the cervix from sensors immediately adjacent to and in contact with the cervix.

An object of the invention is to provide a device for the continuous monitoring of the condition of the cervix while permitting the subject to be completely ambulatory and have complete freedom of action.

Another object of the invention is to provide a device that can be employed in a system wherein the monitoring of the cervix can be done remotely.

A further object of the invention is to continuously monitor physiological changes in the tissue of the cervix employing electrochemical impedance spectroscopy (EIS).

Another object of the invention is to provide a device that can be employed early in pregnancy to detect the early onset of conditions that indicate the likelihood of a preterm birth.

It is an object of the invention to provide a device that can be employed in a system that collects and analyses data and distinguishes between normal conditions of the cervix and relevant physiological changes in cervical tissue that may indicate early stages of labor.

In one preferred mode digitized data is converted to impedance measurements and is conveniently processed to correlate to cervical stromal impedance (CSI) values stored in the database of the interface device. Typical values may be found in Gandhi, et al., “Comparison of Human Uterine Cervical Electrical Impedance Measurements Derived Using Two Tetrapolar Probes of Different Sizes,” European Journal of Obstetrics & Gynecology and Reproductive Biology, Vol. 129, Issue 2, December 2006, 145-149, which is incorporated herein by reference in its entirety.

Alternatively, in another embodiment the processed data is accumulated to generate baseline data for the individual being monitored. The baseline data can be displayed as a curve or displayed in textual form. In either case a significant change in the slope of the curve or an increase or decrease in data values is an indication that a significant change in the physiological condition of the cervix and that labor may be imminent.

In accordance with the invention these techniques can be employed early on in the pregnancy with no discomfort to the individual and without the necessity of restraining or otherwise preventing the free movement of the individual during the monitoring process.

To maintain freedom of movement for the individual being monitored all transmissions of data from the device is wireless, i.e. direct transmission from the device to an external module, such as a computer, which for computation of impedance or indirect to an external device, such as a cell phone, for more robust retransmission to a remote computer for calculating impedance.

These and other objects and advantages will become apparent from the following description of the invention taken in conjunction with the drawings.

### BRIEF DESCRIPTION OF THE DRAWINGS

FIG. 1 is a block diagram of steps using the micro-device of the invention;

FIG. 2 is an isometric view of one embodiment of a device i n accordance with the invention;

FIG. 3 is a cross sectional view of the device o f FIG. 2 positioned adjacent the cervix;

FIG. 4 is an isometric view of an embodiment of the device that is adapted for suturing onto the cervix;

FIG. 5 is an isometric view of another embodiment of the device of the invention illustrating the power supply extending from the body of the device;

FIG. 6 is a cross sectional view of the device o f FIG. 5 positioned adjacent the cervix;

FIG. 7 is an isometric view of an embodiment of the invention in which the device also functions as a constricting pessary;

FIG. 8 is a block diagram of the microcircuit for simulating EIS;

FIG. 9 is an isometric view of the device of FIG. 1 illustrating location of the circuitry, electrodes and antenna; and

FIG. 10 illustrates radiation from the antenna of the device of FIG. 8;

### DESCRIPTION OF THE INVENTION

The system of the invention is suited for monitoring the pregnancy of mammals, however for ease of description the invention will be described in connection with monitoring the pregnancy of a human female. The device of the invention includes circuitry that simulates EIS using low power direct currant.

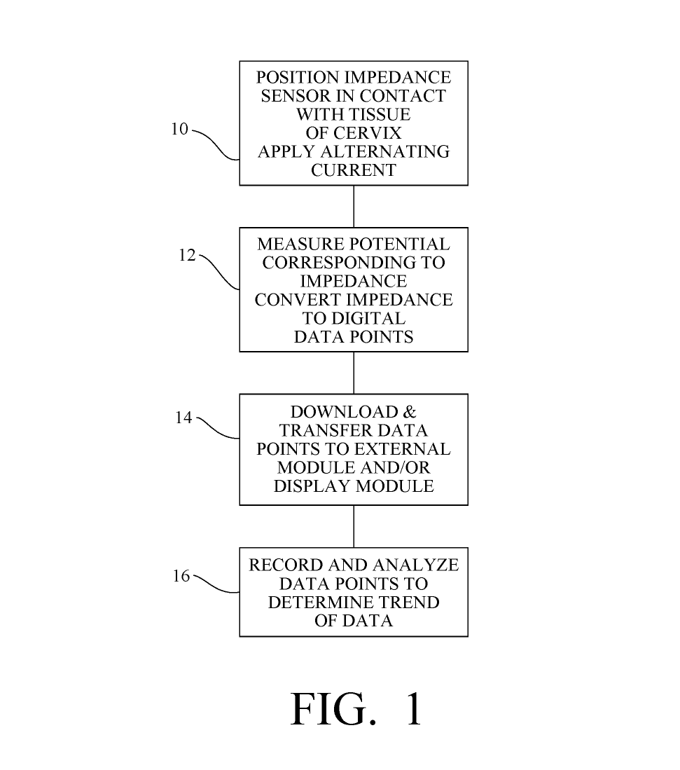

FIG. 1 illustrates the basic steps for utilization of the device of the invention to determine the physiological condition of cervical tissue. The first step shown at 10 is to secure electrodes in intimate contact with cervical tissue. Circuitry is provided to create binary pulses of direct current and to convert the pulses to alternating current. The potential of the pulses passing through the cervical tissue is sensed and digitized at 12 to create digital data points. At 14 the digital data points are wirelessly transmitted to an external module for conversion to impedance data points. The impedance data points may be displayed at the external module or transmitted to a remote display module. The final step 16 is to record and analyze the impedance data to determine a data trend and to treat the patient if necessary, baseline data for the respective parameter. A significant change in received data from the baseline data will cause the software program to transmit a warning to the clinician that some action is required.

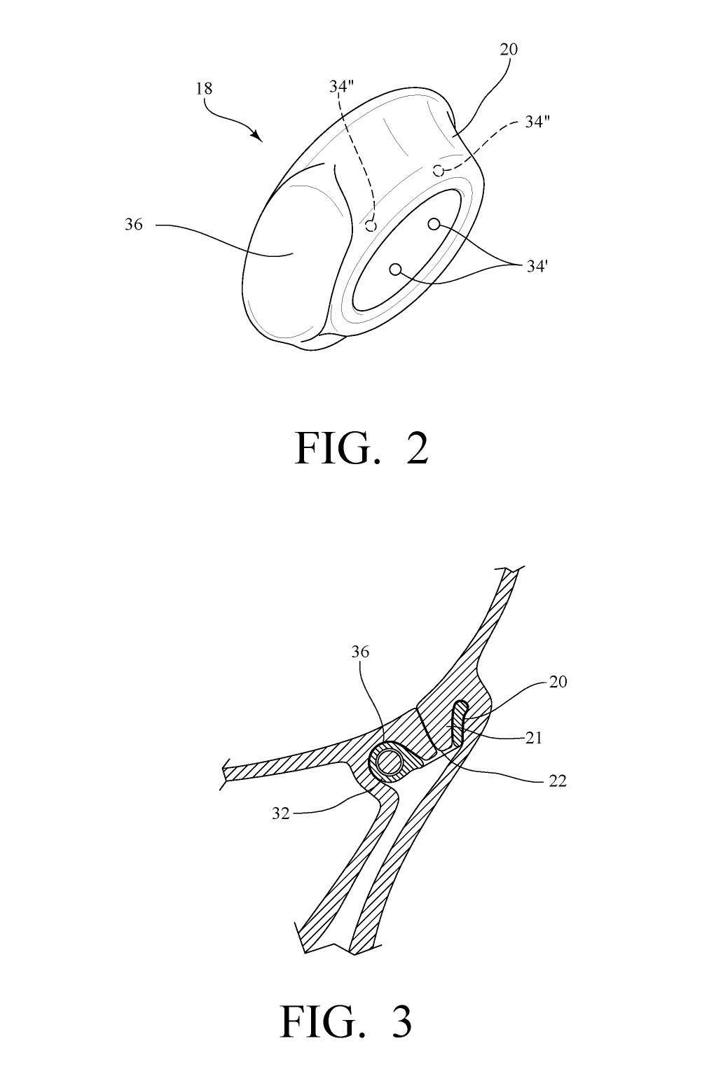

As illustrated in FIG. 2 and FIG. 3, in one embodiment a device 18 for sensing potential through cervical tissue 22 comprises a flexible annulus 20 for positioning around a cervix 22. The annulus 20 is formed of a resilient, electrically non-conductive, biocompatible material, such a s polyurethane, silicone or silicone rubber that exhibits the desired properties of resiliency, flexibility, biocompatibility and non-conductivity. In one embodiment the annulus 20 is transparent or translucent for activation of the device 12 circuitry by a light sensitive on/off switch as will be more fully described in connection with FIG. 8. The inner radial surface of the annulus 20 is provided with one or more Kelvin configured sensing electrodes 34′ and one or more current injection electrode electrodes 34″ that are in electrical communication with signal converting electronics for converting potential measurements to digital data. The annulus 20 being flexible and resilient can be slightly stretched between the fingers of a clinician for placement around the cervix 22. When the clinician's fingers are removed the annulus 20 will return to its original diameter to provide intimate contact between the electrodes 34 and the cervix 22.

In view of the fact that the patient is ambulatory during the monitoring process there exists the danger of a shift in the position of the annulus 20 on the cervix 22 which will result in the change of the position of the electrodes 34′ and 34″. Any such change can affect the applied pressure on the electrodes. Either or both events will change the resulting data which can give a false indication of a change of impedance in the cervix tissue or could hide an impedance change in cervix tissue. Notice of any such shift the annular body 22 can be indicated from an unusual change in the total electrode potential which is the sum of the potential measured between the injecting electrodes 34′ and the pair of electrodes 34″ that are measuring cervix tissue potential. Such an unusual change in the total potential correlates to an unusual change in total impedance and will clearly show in the displayed data trend. Such a change in the data trend may also be an indication of a system malfunction. As a precaution, however, depending on the predicted activity of the patient, the device 18 can be adapted for stitching on to the cervix 21 to prevent any shifting.

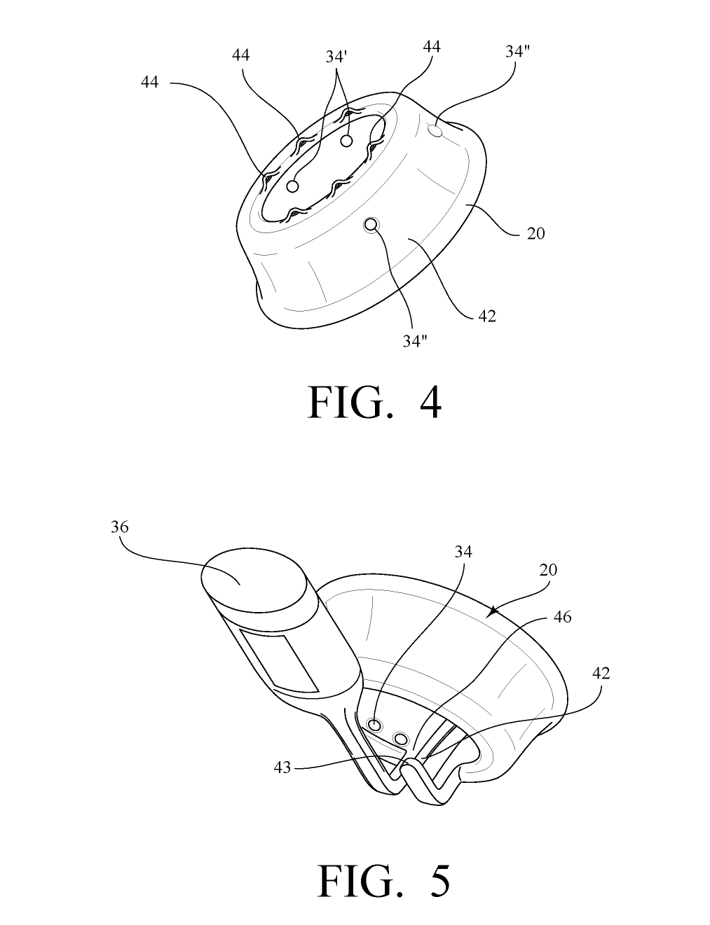

FIG. 4 illustrates an embodiment of the device 18 comprising the annulus 20, electrodes 34′ and 34″ and circuitry as described in FIG. 1. To further secure the device 18, a plurality of eyes 44 are formed on a circumference of the annulus 20, as shown, for suturing the annulus to the patient's cervix.

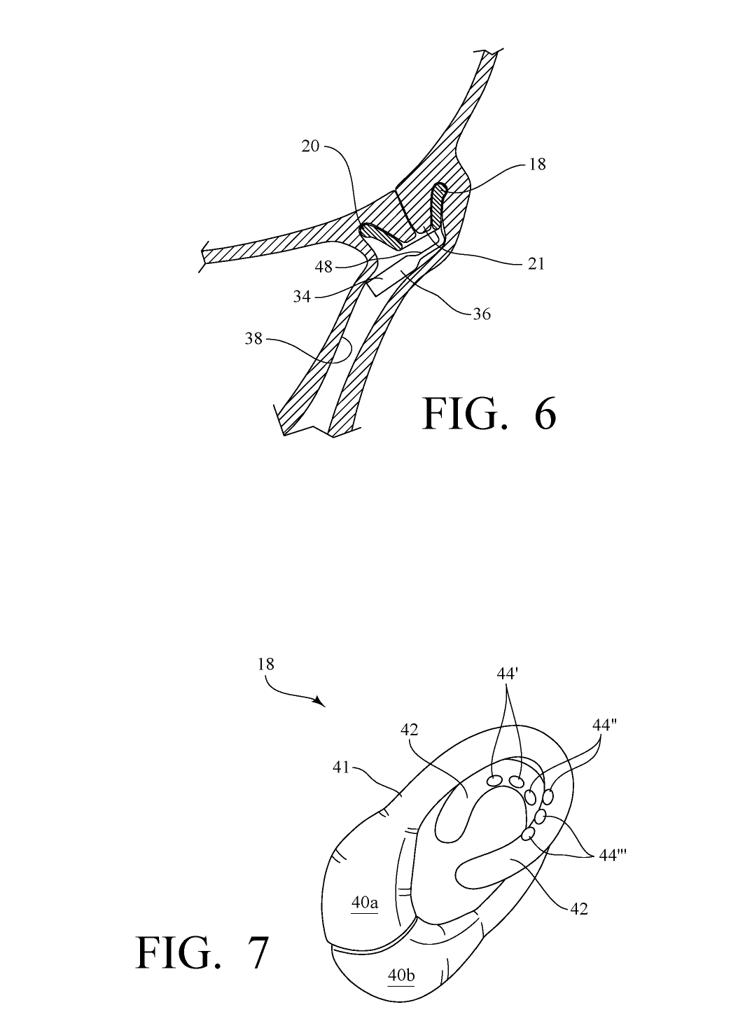

Yet another embodiment of the device, shown generally as 18, is illustrated in FIG. 5 and FIG. 6 in which the annular body 20 is interrupted to define free ends 42 and 43. Free end 42 extends away from the annular body 22 and is elongated to define a compartment 36 for battery and associated electronics. A single pair of electrodes 34 serves both as the injecting electrodes and the measuring electrodes. The device functions as described above in measuring impedance. However, as shown in FIG. 6 when the annulus 20 is disposed on the cervix 22 the compartment 36 is urged against the uterine wall 38 as an aid in maintaining the annulus in position on the cervix.

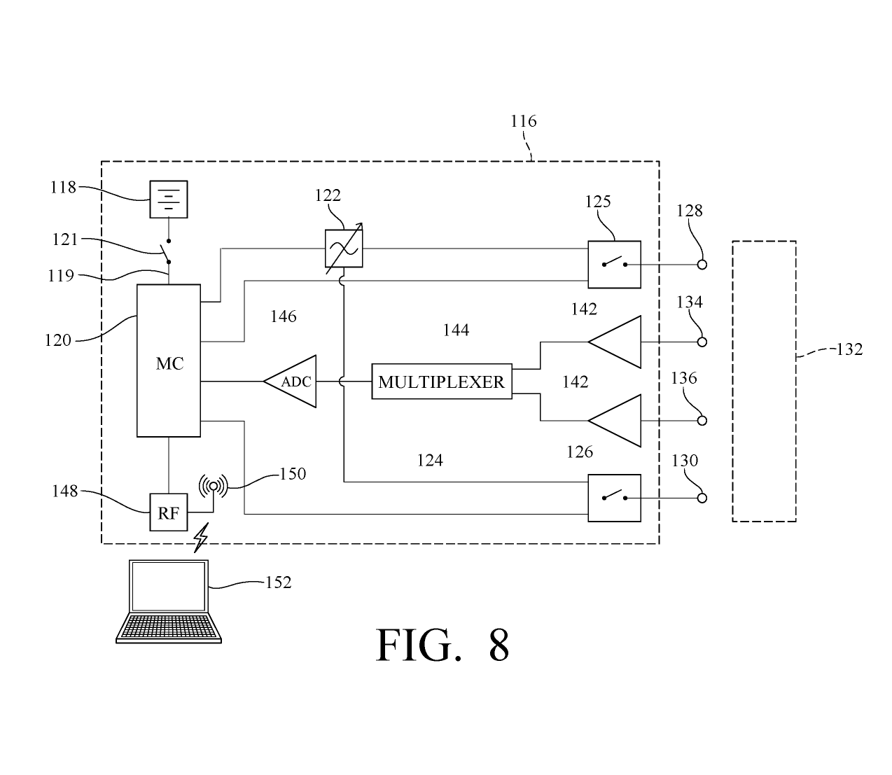

In another embodiment shown in FIG. 7 the device 18 is configured as a constricting pessary to maintain closure of the cervix 21. In this embodiment the device 18 comprises the annular body 22 on which is formed enlarged compartments 40a and 40b in which are respectively disposed the battery and the associated electronics. The annular body 22 is interrupted to define a pair of resilient, flexible U-shaped arms 42. In this embodiment electrodes 44′, 44″ and 44′″ are disposed in the arms 42. The electrodes 44′ and 44′″ comprises the injecting electrodes and electrode pair 44″ comprise the impedance measuring electrodes.

The device 18 is positioned around the cervix by spreading the U-shaped arms 42.

Once positioned, the arms 42 are allowed to return to their original configuration so that each arm securely contacts the cervix to provide constrictive support of the cervix as a pessary and to maintain the electrodes 44′, 44″ and 44′″ securely positioned in intimate contact with the cervix.

The circuitry utilized in the device of the invention must be able to supply alternating currant to the injection electrodes in order to simulate EIS. In addition, the circuitry power requirements must be low for the device to function for relatively long periods. Finally, the circuit must be miniaturized in order to fit in a small body such as a pessary. Conventional EIS circuitry cannot be used in the present invention as it requires too many components and cannot be miniaturized to fit in a pessary. Also, EIS requires a constant current source and too much power to apply a constant source of alternating current to the biological load. In accordance with the invention, Kelvin configured circuitry is described that can be miniaturized to about size of a quarter to fit in a pessary. The micro-circuit operates on low power direct current which it converts to sinusoidal binary pulses so that the device of the invention operates as though it were measuring tissue impedance by EIS.

FIG. 8 a block diagram of a micro-circuit 116 that simulates EIS and which miniaturized to fit in a small body such as pessary. A low power battery 118 electrically communicates through line 119 to a microcontroller 120. An on/off switch 121 is disposed in the line 119 for activating and deactivating the circuit. Because the micro-circuit is embedded in a body such as a pessary it is preferred that the body be composed of a material that is at least translucent and switch 121 be light activated so the circuitry can be activated just prior to application of the micro-device. Other means for activating the on/off switch 121 may be employed, such as sound, if a translucent or transparent pessary is undesirable.

A microinverter 122 communicates with positive analog switches 125 through line 123 and with negative analog switches 126 through negative line 124. Switches 125 communicate with positive injection electrode 128 and switches 126 with negative injection electrode 130. As illustrated, common is at negative electrode 130 so that the polarity is positive and the positive injection electrode injects a positive current pulse into tissue 132. The microcontroller 120 changes common to the positive electrode 128 reversing polarity and closing switch 126 for injection of a negative current pulse through electrode 130. The microcontroller 120 controls switch 125 and 126 to rapidly open and close to inject alternate positive and negative pulses of current in to tissue 132. In this manner alternating current is produced and EIS is simulated. Present day micro-controllers such as the Texas Instruments MSP430 series have become quite competent with peripheral resources and can handle the rapid multiple switching operations.

The potential of the alternating current through tissue 132 is sensed by electrodes 134 and 136 and transmitted through lines to amplifiers 142 and multiplexor 144 and analog to digital converter 146. The digitized potential signal is transmitted to microcontroller 120 and is wirelessly transmitted by R/F circuitry 148 including a circular antenna 150 to an external module 152. The R/F transmitter circuitry uses as low a frequency and power level as practical to minimize any possibility of fetal harm. The R/F circuitry may operate in the range of about 300 MHz to about 900 MHz without the danger of harming the patient or the fetus. A frequency in the 400 MHz band and a power level of about −30 dBm provides a connectivity range of greater than three meters which is sufficient and which ensures the safety of the fetus. The circuits are maintained at a very low power level until awakened by the micro-controller-based firmware. The unprocessed data is uploaded at regular intervals to the external module 152.

The external module 152 may be an external interface device, such as a cell phone, which is carried by or in close proximity to the patient for more robust transmission to a remote computer or may be directly transmitted to a computer if it is within the transmission range of the RF transmission from the micro-device 18. At the computer impedance is calculated from the digitized data, the positive and negative impedances are averaged and read out or displayed to produce an impedance trend line for the patient. By averaging a “positively driven” impedance and a “negatively driven” impedance and taking the average, an unbiased impedance value may be derived.

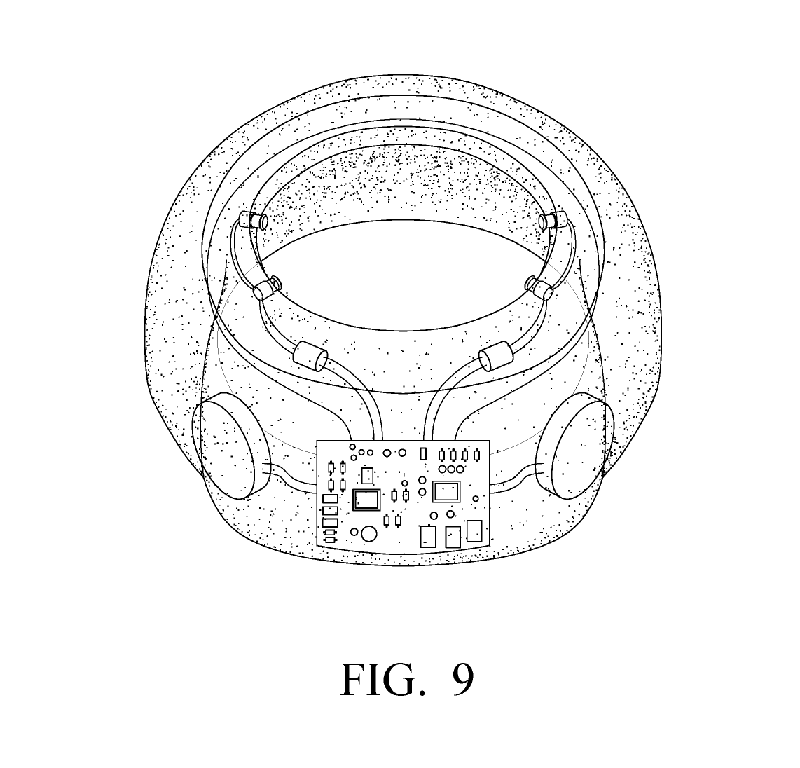

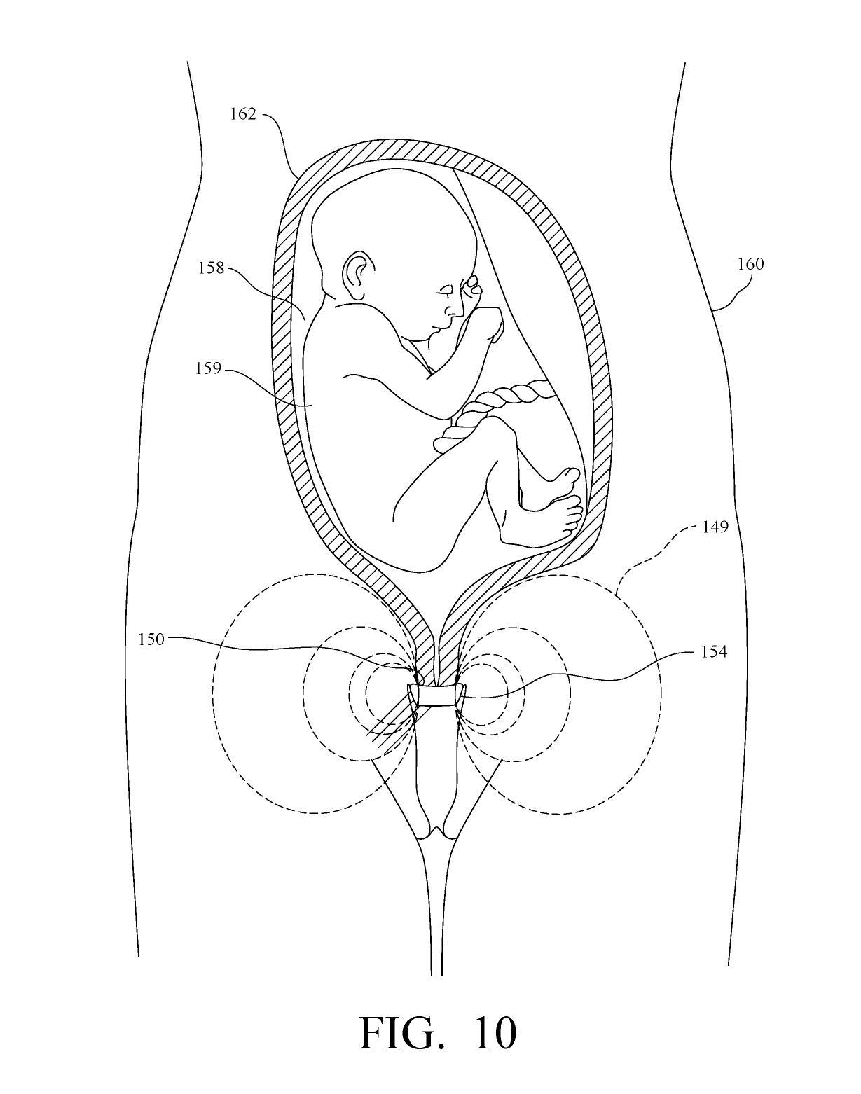

FIG. 9 illustrates a preferred embodiment of the device 18 in which like numbers represent like parts and like functions. The device 18 comprises a pessary 154 in which is embedded the circuitry 116 of FIG. 8. Preferably the pessary 154 is composed of a bio-compatible translucent material and includes the on/off switch 121 that is light activated after the circuitry 116 is embedded in the pessary. The low power battery source consists of a pair of 1.5 v batteries 118. Injection electrodes 128 and 130 and sensing electrodes 134 and 136 are in electrical communication with the circuitry 116 and are disposed on the inner wall of the pessary 154 for contact with the cervical tissue.

As discussed above the digitized unprocessed data is transmitted by R/F to an external module for calculation of impedance or to an external interface module for retransmission to a more remote module, such as a computer, for calculating and displaying the impedance measurements. The R/F circuitry includes the circular antenna 150, comprising an antenna wire, extending around the circumference of the pessary 154

There is evidence that electromagnetic radiation from sources such as high tension power lines, television sets, appliances, R/F transmission systems and the like can be harmful to biological organisms. A fetus may be highly susceptible to injury from electromagnetic radiation from R/F transmission. Thus, radiation due to the R/F transmission of data must be taken into account to avoid possible injury to the fetus particularly since the device 18 is designed to be worn for extended periods of time while the patient's cervix is being monitored.

FIG. 10 illustrates a fetus 156 disposed within the uterus 158 of a female patient 160. The uterus 158 is defined by a uterine wall 162 which is located in the abdomen 164 of the patient 158. Extending ventrally from the uterus 158 is the cervix 164 which normally closes the uterus 158 but the tissue of which under goes changes such as effacement and ripeness during the birthing process. These changes are conventionally detected by manual examination as the patient is starting into labor. The micro-device of the invention utilizes EIS simulating micro-circuitry as described in FIG. 8 to monitor the cervical tissue and detect physiological changes to the cervix as an early indicator of impending labor before such changes can be detected by examination of the patient. The micro-device 18 is placed about the cervix 164 of the patient 158 and does not interfere with the normal activities of the patient. In addition, the clinician can monitor the physiological condition of the cervical tissue without the need of patient's presence at the clinic or office.

As illustrated the micro-device 18 in the form of a pessary 154 as described in FIG. 9 fits snugly about the cervix 164 and holds the positive and negative injection electrodes 126 and 128 and the sensing electrodes 134 and 136 in intimate contact with the tissue of the cervix 164. Two 1.5v batteries 118 embedded in the micro-device 18 provide low power direct current to the circuitry 116 that converts the current into positive and negative sine waves to simulate alternating current as required by EIS. The potentials of the positive and negative sine waves are detected by the sensing electrodes 134 and 136 and digitized as described in connection with FIG. 8. The digitized data are wirelessly transmitted for calculation of the impedance of the cervical tissue. During transmission an electromagnetic radiation field is created around the antenna wire comprising the circular antenna 150. Due to the configuration of the circular antenna 150 the electromagnetic radiation field is created around the antenna wire and is directed away from the fetus 156.

While various embodiments have been described above, it should be understood that they have been presented by way of example only, and not limitation. Thus, the breadth and scope of a preferred embodiment should not be limited by any of the above-described exemplary embodiments, but should be defined only in accordance with the following claims.

1. A micro-device for simulating electrochemical impedance spectroscopy comprising:
a. a flexible, resilient, non-conductive annular body having a contact surface for contact with a biological load;
b. a source of low power direct current disposed in said body;
c. micro-circuitry including a microinverter and a micro-controller disposed in said body for conversion of said low power direct current to alternating current and for sequentially alternating circuit polarity;
d. said body contact surface having at least two electrodes in communication with said micro-circuitry for contact with said biological load; and
e. wireless transmission circuitry.

2. The micro-device of claim 1 wherein said body is a pessary and said source of low power direct current and said micro-circuitry are disposed in said pessary.

3. The micro-device of claim 1 wherein said source of low power direct current is a 3v battery.

4. The micro-device of claim 1 wherein said source of low power direct current are two 1.5v batteries.

5. The micro-device of claim 1 wherein said micro-controller alternately switches polarity of said circuitry to alternately transmit positive and negative current.

6. The micro-device of claim 1 further including at least one analog switch controlled by said micro-controller that rapidly opens and closes when said circuitry is positive and opens and closes when said circuitry is negative so that alternate pulses of positive and negative current are injected into said biological load to simulate alternating current,

7. The micro-device of claim 2 wherein the composition of said body is at least translucent and said micro-circuitry includes a light sensitive switch for activation of said circuitry after said micro-circuitry is disposed in said pessary.

8. The micro-device of claim 1 wherein said wireless transmission includes a circular antenna.

9. The micro-device of claim 8 wherein said circular antenna comprises an antenna wire extending around the circumference of said annular body.

10. The micro-device of claim 1 wherein said body contact surface includes two injection electrodes, one said injection electrode for the introduction of positive current into said biological load and one other said injection electrode for the introduction of negative current into said biological load, said positive and negative current being alternately introduced as pulses.

11. The micro-device of claim 1 wherein said body contact surface includes two sensing electrodes for measuring the potential of positive and negative current pulses flowing through said biological load.

12. The circuit of claim 1 embedded in a pessary for monitoring the physiological condition of cervical tissue.

13. Micro-circuitry that simulates electrochemical impedance spectroscopy comprising a low power source of direct current, a microinverter for converting direct current to sine-wave current, a micro-controller for alternately activating a positive analog switch and a negative analog switch to create pulses of said sine-wave current, said micro-controller alternately reversing the polarity of said micro-circuitry to produce alternate positive and negative current pulses, a positive injection electrode for injecting said positive current pulses into a biological load and a negative injection electrode for injecting negative current pulses into said biological load, a pair of sensing electrodes for sensing the potentials of said positive and negative current pulses in said biological load and for producing signals in response to said sensed potentials, an amplifier in electrical communication with each said sensing electrodes for amplifying said signals, an analog to digital converter for digitizing said amplified signals and transmitting said digitized signals to said micro-controller, said micro-circuitry further including R/F circuitry including a circular antenna for transmitting said digitized signals to an external module.

14. A system for monitoring the condition of a pregnant mammal by monitoring the physiological condition of the cervix comprising a flexible, electrically non-conductive biocompatible device adapted to be securely positioned on the cervix, said body defining an inner surface for contact with said cervix, micro-circuitry disposed in said device comprising a low power source of direct current, a microinverter for converting direct current to sine-wave current, a micro-controller for alternately activating a positive analog switch and a negative analog switch to create pulses of said sine-wave current, said micro-controller alternately reversing the polarity of said micro-circuitry to produce alternate positive and negative current pulses; said inner surface including a positive injection electrode for injecting said positive current pulses into said cervix and a negative injection electrode for injecting negative current pulses into said cervix in response to said micro-controller, a pair of sensing electrodes for sensing the potentials of said positive and negative current pulses in said cervix and for producing signals in response to said sensed potentials, an amplifier in electrical communication with each said sensing electrode for amplifying said signals, an analog to digital converter for digitizing said amplified signals and transmitting said digitized signals to said micro-controller, transmission circuitry in association with said micro-controller for direct or indirect transmission to an external module for computation and display of impedance, said transmission circuitry including a circular antenna and a computer for calculating impedance from said digitized signals and for displaying said impedance, whereby the physiological condition of an individual's cervix can be remotely monitored while the individual being monitored retains full freedom of activity.
